(19) United States
(12) Patent Application Publication
(10) Pub. No.: US 2017/0218447 A1
(43) Pub. Date: Aug. 3, 2017

(54) PLATFORM INDEPENDENT HAPLOTYPE IDENTIFICATION AND USE IN ULTRASENSITIVE DNA DETECTION

(71) Applicants: THE JOHNS HOPKINS UNIVERSITY, Baltimore, MD (US); KENNEDY KRIEGER INSTITUTE, INC., Baltimore, MD (US)

(72) Inventors: James R. Eshleman, Lutherville, MD (US); Sarah J. Wheelan, Baltimore, MD (US); Jonathan Pevsner, Baltimore, MD (US)

(21) Appl. No.: 15/500,736

(22) PCT Filed: Aug. 5, 2015
(86) PCT No.: PCT/US2015/043748

§ 371 Date: Jan. 31, 2017

Related U.S. Application Data
(60) Provisional application No. 62/033,254, filed on Aug. 5, 2014.

(51) Int. Cl. C12Q 1/68

(52) U.S. Cl. CPC C12Q 1/6881; C12Q 1/6858; C12Q 2600/156; C12Q 2600/172

(57) ABSTRACT

The present invention provides methods for analyzing blocks of closely spaced SNPs, or haplotypes for use in identification of the origin of DNA in a sample. The methods comprise aligning common alleles of a gene of interest and identifying a region containing a plurality of SNPs which is flanked by non-polymorphic DNA which can be used for primer placement. Any sequencing method, including next generation sequencing methods can then be used to determine the haplotypes in the sample with a lower limit of detection of at least 0.01%. These inventive methods are useful, for example, for identification of hematopoietic stem cell transplantation patients destined to relapse, microchimerism associated with solid organ transplantation, detection of solid organ transplant rejection by detecting donor DNA in recipient plasma, forensic applications, and patient identification.

### REFERENCE TO RELATED APPLICATIONS

This application claims the benefit of U.S. Provisional Patent Application No. 62/033,254, filed on Aug. 5, 2014, which is hereby incorporated by reference for all purposes as if fully set forth herein.

### INCORPORATION-BY-REFERENCE OF MATERIAL SUBMITTED ELECTRONICALLY

The instant application contains a Sequence Listing which has been submitted in ASCII format via EFS-Web and is hereby incorporated by reference in its entirety. Said ASCII copy, created on Aug. 5, 2014, is named P12978-01_ST25.txt and is 641 bytes in size.

### BACKGROUND OF THE INVENTION

Myeloablative conditioning and allogeneic stem cell transplantation (alloSCT) has historically been limited to the treatment of lethal hematologic malignancies in children or young adults. More recently with the advent of highly immunosuppressive, non-myeloablative regimens, the clinical use of alloSCT has expanded to include older, less fit patients with hematologic malignancies as well as patients with non-malignant disorders such as sickle cell disease (SCD). Non-myeloablative conditioning regimens offer the additional safeguard of recovery of autologous hematopoiesis in the event of graft rejection and so may be a safer option in patients at risk for immune mediated rejection of the donor graft.

Chimerism testing at set intervals is an effective method for detecting graft rejection or recurrence of the original hematopoietic neoplasm after allogeneic HSCT (with either bone marrow or peripheral blood stem cells). Decades ago, bone marrow engraftment monitoring was performed using Southern blotting and minisatellite or Variable Number of Tandem Repeats (VNTR) loci. Today, short tandem repeat (STR), or microsatellite, loci are most commonly used for this purpose. STRs are composed of 10-60 tandemly repeated units, where each unit is 1-6 bases in length. They are widely distributed throughout the human genome, are highly variable between individuals, and therefore allow for excellent differentiation between individuals, including patient and donor, even if they are closely related. Most laboratories use multiplex PCR based kits, originally developed for forensics analysis using Combined DNA Index System (CODIS) loci. STR analysis most commonly involves PCR amplification using fluorescently labeled primers, followed by amplicon separation by capillary electrophoresis.

Other polymorphic DNA that could be used to monitor bone marrow engraftment include single nucleotide polymorphisms (SNPs). SNPs are theoretically superior to STR based analyses because analysis of STR loci by capillary electrophoresis is relatively insensitive (limit of detection 1-5%) and microsatellite alleles of varying length amplify with different efficiencies, thus making them inherently biased. STR amplification can also be difficult in the setting of highly degraded DNA. However, SNPs are less attractive as targets due to their inherently lower informativity (e.g. only two possible bases for a bi-allelic SNP vs. 10 or more alleles for some microsatellites), requiring many more SNPs to be tested to identify those that distinguish donor from recipient. For example, we previously estimated that one would need to screen more than 20-30 individual SNPs to confidently identify one SNP where the donor is homozygous for one allele and an unrelated recipient is homozygous for the other. Fewer would need to be included if heterozygotes were included, but more would have to be analyzed for related individuals.

Recently emerging next generation sequencing (NGS) technologies along with their decreasing costs are now feasible for clinical testing. However, all NGS technologies currently have high error rates, in the range of 0.04%-1% at each base, which precludes their use for ultrasensitive detection of a single SNP. One solution to this problem is sequencing blocks of closely spaced SNPs, i.e. haplotypes. Haplotypes are regions of the genome where polymorphic areas are sufficiently close that they are inherited together, including either genes (e.g. HLA-1 A, HLA-B, etc) within a locus, or multiple SNPs within a region of DNA.

### SUMMARY OF THE INVENTION

In the present invention, the inventors first used the HLA-A locus as proof-of-principle to demonstrate a novel, inventive approach which permits high sensitivity, precision, and accuracy. These methods were then used to study bone marrow (BM) samples from a cohort of patients who engrafted after HSCT and tested as all donor by STRs, and found that low level patient DNA is commonly present. To identify additional loci that could be used for this purpose, the inventors used the inventive methods to comprehensively analyze the human genome and identified other regions with highly informative haplotypes. These inventive methods can be used in many other situations where routine haplotyping of patient samples would improve patient safety.

In accordance with an embodiment, the present invention provides a method for identifying informative haplotypes useful for identity testing comprising: a) obtaining the DNA sequences of a plurality of individual genomes of a mammal; b) identifying within the genomes of a) haplotypes comprising both allelic DNA sequences of about 100 to 400 or more base pairs in length, having at least one or more polymorphic regions which are flanked at both the 5′ and 3′ ends with constant regions of at least about 20 base pairs in length; c) identifying within the haplotypes of b) those haplotypes which have at least about 2 or more single nucleotide polymorphism variants of the polymorphic regions; and d) identifying those haplotypes of c) as informative if at least 1 haplotype from a first individual genome has at least 2 or more single nucleotide polymorphism differences from both alleles of a second individual genome.

In accordance with another embodiment, the present invention provides a method for determining the DNA sequence of one or more informative haplotypes in a DNA sample of a mammal comprising: a) obtaining a sample containing a sufficient amount of DNA which comprises at least about 100 to about 100,000 genomes of the mammal; b) purifying the DNA from a); c) amplifying the DNA from b) using PCR and primers and probes specific for one or more informative haplotypes and for the sequencing method (including Sanger sequencing, pyrosequencing, etc.) being used to analyze the DNA sequences of the informative haplotypes; d) analyzing the plurality of DNA sequences of the amplified informative haplotypes for single nucleotide polymorphisms in c); e) comparing the DNA sequence single nucleotide polymorphisms found in d) to the DNA sequence single nucleotide polymorphisms for one or more reference informative haplotypes, wherein when a DNA sequence of the one or more informative haplotypes from d) does not contain all of the single nucleotide polymorphisms of the one or more reference informative haplotypes, the DNA sequence is discarded as erroneous; and f) establishing that when a DNA sequence of the one or more informative haplotypes from d) contains all of the single nucleotide polymorphisms of the one or more reference informative haplotypes, the DNA sequence is a match and the haplotype identity is confirmed.

### BRIEF DESCRIPTION OF THE DRAWINGS

FIGS. 1A-1D depict an embodiment of the haplotype identification methods of the present invention. (1A) Shown is a theoretical locus containing 4 possible SNPs, where the donor (left) is homozygous adenine at each of the SNPs for both alleles and the patient (recipient, right) is homozygous cytosine for both alleles. (1B) In a post-transplant sample, one detects 10,000 reads that perfectly match the donor genotype, 10 reads with a cytosine (yellow) at the third SNP and 10 reads with a cytosine at the first SNP (yellow). The 20 reads with a single cytosine can be interpreted as PCR errors since they do not perfectly match either donor or patient alleles. Accordingly, these results would be interpreted as 100% donor. (1C) Another post-transplant sample contains 9,900 reads that perfectly match the donor haplotype, while 100 reads perfectly match the patient haplotype. Accordingly this sample is 1% patient. (1D) The distal region of HLA-A exon 3 (blue rectangle) and intron 3 (blue line) containing 18 potential SNPs (indicated with “X”s) and two HLA-A PCR primers (yellow rectangles) as shown. Note that the PCR primers are tailed with adapters (orange) for the DNA sequencing primer to bind (A-adapter) or to covalently anchor the amplicon onto the bead (P1-adapter). The total length of the HLA targeted region is 245 bp and the total length of the amplicon is 298 bp, including the adapters. Not shown is the 4-base library key located between the A-adaptor and the forward HLA-A primer.

FIG. 2 depicts numbers of discriminating SNPs between common HLA-A alleles. Common European-origin HLA-A alleles were collected, aligned, and number of discriminating SNPs between them determined. The homozygous genotypes analyzed in FIGS. 3, A*01 and A*02, differ by 11 SNPs (double box). The genotypes from the dilution series in FIG. 4 are A*01/A*02 into A*02/A*1 24, where the unique alleles vary by 7 SNPs (bold box). Other combinations vary by fewer SNPs, such as A*68 vs. A*02 (dashed box). Included are only alleles that occur at 1% or higher in the European origin population.

FIGS. 3A-3C show molecular specificity of the inventive methods. Samples homozygous for the A*01 allele (panel 3A) and the A*02 allele (panel 3B) were PCR amplified and NGS sequenced. Each was analyzed for the presence of the other allele and for hybrid molecules containing one or more SNPs of the other allele. (3C) “Waterfall” plot of the percent reads for each combination of the eleven SNP differences for the two samples. For example, the “10A1 +1A2” bar reflects the mean number of erroneous reads (9/11=0.82), in the pure A*01 sample where any one of the 11 SNP positions matches the A*02 allele (0.82/201,605, from panel 3A). Likewise the “9A1 +2A2” bar reflects the lack of any reads in the pure A*01 sample where 2 adjacent SNPs match the A*02 allele at any of the positions.

FIGS. 4A-4B show the linearity of using the inventive methods for NGS haplotyping and microsatellite analysis. (4A) Linearity of the SNP haplotyping approach. A*01/A*02 cells were serially diluted into A*02/A*24 cells, DNA isolated, and tested by HLA-A NGS. NGS HLA-A*01 counts expressed as a percentage, as a function of the percent HLA-A*01 bearing cells. Error bars: +/−1 SEM. The error bar for 0.1% bearing cells is too small to be seen. The error bar for 0.003% is absent as only 1 out of 2 replicates amplified. Note that the NGS haplotyping data begins at 1% and is serially diluted down 1 from there. (4B) Post-transplant patient samples with various percentage of patient DNA were analyzed using the Profiler and Identifiler kits. Note the lack of the ability to detect DNA below the limit of detection of 3%.

FIG. 5 depicts the identification of patient DNA in bone marrow samples. Unique patient DNA was detected in BM samples, which demonstrated all donor by conventional STR assays, after HSCT.

FIGS. 6A-6B show that HCT116 cells were serially diluted into DLD-1 cells and DNA co-isolated. HLA-A based haplotype counting was used to determine the true concentration (x-axis) and mutations detected in the samples using the AmpliSeq panel (Ion Torrent, Life Technologies, panel 6A) or ddPCR (Bio-Rad, panel 6B).

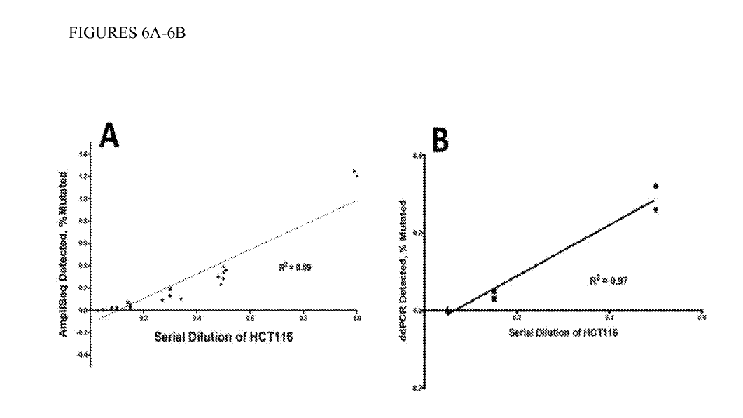

FIG. 7 depicts donor molecules per million as a function of time after bone marrow infusion in a patient following limb transplantation.

FIG. 8 is a graph showing Non-Relapse vs Relapse bone marrow transplant patients detected by haplotype counting. For relapse patients, the sample tested was immediately prior to that which tested positive by STR analysis. For non-relapse patients, the sample tested was followed by 291 days on average and remained fully engrafted.

FIG. 9 shows alignments of novel FARP1 alleles illustrate SNP positions (red) that either match or vary from the reference genome (FARP1-1). We have identified 5 new haplotypes (FARP1-2 to FARP1-6) where bases that deviate from reference are highlighted in yellow.

FIG. 10 depicts the haplotypes of FARP1 locus compared. This figure demonstrates the number of SNPs that vary between any two FARP1 haplotypes. For example, if comparing 2 individuals, one homozygous for FARP1-5 and another homozygous for FARP1-3, these will vary by 8 SNPs (highlighted in bold).

### DETAILED DESCRIPTION OF THE INVENTION

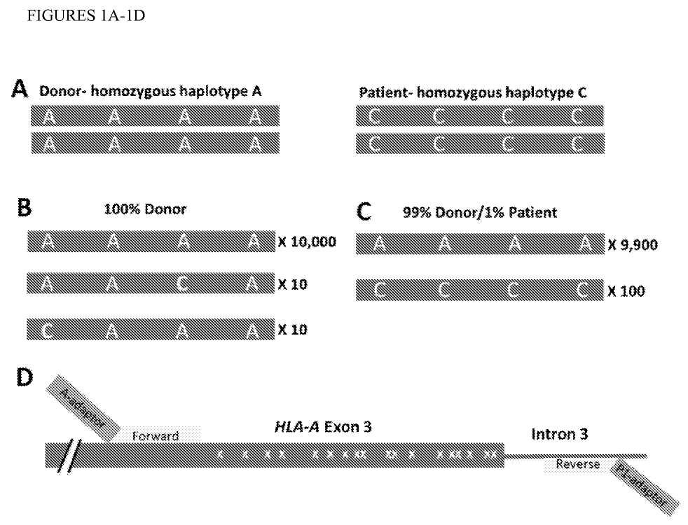

A general schematic of an embodiment of the inventive methods for determining the DNA sequence of one or more informative haplotypes in a DNA sample of a mammal is demonstrated in FIG. 1. Imagine a region of a gene that contains 4 SNPs, and two individuals: a donor who is homozygous for adenine at all 4 SNPs (designated homozygous haplotype A), and a patient (recipient) who is homozygous for cytosine at all 4 SNPs (homozygous haplotype C, FIG. 1A). In a sample that is pure donor (top bar, 10,000 reads), due to the high error rates of NGS, mutations will be seen by NGS, some of which will coincidentally occur at SNP positions (FIG. 1B, bottom 2 bars, 10 reads each). Since these involve only 1 of the 4 SNPs, these can be attributed as PCR errors and discarded since these molecules do not match either the donor or patient alleles. Note that if the strategy used only a single SNP, instead of a haplotype, one would be unable to distinguish between 10 true patient reads vs. 10 PCR error reads. In contrast, if 100 molecules are detected in the post-transplant sample with cytosine at all 4 SNPs, this would indicate the presence of true patient-specific DNA (FIG. 1C).

By “nucleic acid” as used herein includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide.

The nucleic acids used as primers in embodiments of the present invention can be constructed based on chemical synthesis and/or enzymatic ligation reactions using procedures known in the art. See, for example, Sambrook et al. (eds.), Molecular Cloning, A Laboratory Manual, 3^{rd} Edition, Cold Spring Harbor Laboratory Press, New York (2001) and Ausubel et al., Current Protocols in Molecular Biology, Greene Publishing Associates and John Wiley & Sons, NY (1994). For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N⁶-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be purchased from companies, such as Macromolecular Resources (Fort Collins, Colo.) and Synthegen (Houston, Tex.).

The nucleotide sequences used herein are those which hybridize under stringent conditions preferably hybridizes under high stringency conditions. By “high stringency conditions” is meant that the nucleotide sequence specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-10 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C.

“Probe” as used herein may mean an oligonucleotide capable of binding to a target nucleic acid of complementary sequence through one or more types of chemical bonds, usually through complementary base pairing, usually through hydrogen bond formation. Probes may bind target sequences lacking complete complementarity with the probe sequence depending upon the stringency of the hybridization conditions. There may be any number of base pair mismatches which will interfere with hybridization between the target sequence and the single stranded nucleic acids described herein. However, if the number of mutations is so great that no hybridization can occur under even the least stringent of hybridization conditions, the sequence is not a complementary target sequence. A probe may be single stranded or partially single and partially double stranded. The strandedness of the probe is dictated by the structure, composition, and properties of the target sequence. Probes may be directly labeled or indirectly labeled such as with biotin to which a streptavidin complex may later bind.

In accordance with one or more embodiments of the present invention, it will be understood that the term “biological sample” or “biological fluid” includes, but is not limited to, any quantity of a substance from a living or formerly living patient or mammal. Such substances include, but are not limited to, blood, serum, plasma, urine, cells, organs, tissues, bone, bone marrow, lymph, lymph nodes, synovial tissue, chondrocytes, synovial macrophages, endothelial cells, and skin. In a preferred embodiment the biological sample is a breast tissue sample, and more preferably, a breast tumor tissue sample. In other embodiments, the samples can be derived from potential donor organs for use in tissue typing for transplantation, for example, kidney, heart and other organs can be sampled and tested using the inventive methods.

In accordance with an embodiment, the present invention provides a method for identifying informative haplotypes useful for identity testing comprising: a) obtaining the DNA sequences of a plurality of individual genomes of a mammal; b) identifying within the genomes of a) haplotypes comprising both allelic DNA sequences of about 100 to 400 or more base pairs in length, having at least one or more polymorphic regions which are flanked at both the 5′ and 3′ ends with constant regions of at least about 20 base pairs in length; c) identifying within the haplotypes of b) those haplotypes which have at least about 2 or more single nucleotide polymorphism variants of the polymorphic regions; and d) identifying those haplotypes of c) as informative if at least 1 haplotype from a first individual genome has at least 2 or more single nucleotide polymorphism differences from both alleles of a second individual genome.

As used herein, the term “single nucleotide polymorphism or SNP” means a DNA sequence variation occurring commonly within a population (e.g. 1%) in which a single nucleotide—A, T, C or G—in the genome (or other shared sequence) differs between members of a biological species or paired chromosomes. For example, two sequenced DNA fragments from different individuals, AAGCCTA to AAGCTTA, may contain a difference in a single nucleotide. In this case this would be defined as two alleles. The vast majority of common SNPs have only two alleles. The genomic distribution of SNPs is not homogenous; SNPs occur in non-coding regions more frequently than in coding regions or, in general, where natural selection is acting and fixating the allele of the SNP that constitutes the most favorable genetic adaptation. Other factors, like genetic recombination and mutation rate, can also determine SNP density. In accordance with the present invention, the inventive methods are able to detect two or more SNPs between haplotypes of a specific gene locus of interest.

As used herein, the term “haplotype” means an allelic DNA sequence of interest having about 100 to 400 or more base pairs in length, and having at least one or more polymorphic regions which are flanked at both the 5′ and 3′ ends with constant regions of at least about 20 base pairs in length; and wherein the haplotypes have at least about 1 or 2 or more single nucleotide polymorphism variants of the polymorphic regions.

The term “constant region” means the portion of the haplotype sequence where there are no polymorphisms or SNPs found.

As used herein, the term “next generation sequencing, or NGS” refers to a number of different modern sequencing technologies including Illumina (Solexa, MiSeq, HiSeq and NextSeq) sequencing, Roche 454 sequencing, Ion torrent: Proton/PGM sequencing, and ABI/Life Technologies SOLiD sequencing, Oxford Nanopore sequencing, Pacific Biosciences'RS II (Menlo Park, Calif.) which are known in the art.

As used herein, it will be understood that any high-throughput technique for sequencing nucleic acids can be used in the methods of the present invention. DNA sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLiD sequencing. Sequencing of the separated molecules has more recently been demonstrated by sequential or single extension reactions using polymerases or ligases as well as by single or sequential differential hybridizations with libraries of probes. These reactions have been performed on many clonal sequences in parallel including demonstrations in current commercial applications of over 100 million sequences in parallel. These sequencing approaches can thus be used to study the identified haplotypes of the present invention in any gene locus of interest.

In an embodiment of the present invention, high-throughput methods of sequencing are employed that comprise a step of spatially isolating individual molecules on a solid surface where they are sequenced in parallel. Such solid surfaces may include nonporous surfaces (such as in Solexa sequencing, e.g. Bentley et al, Nature, 456: 53-59 (2008) or Complete Genomics sequencing, e.g. Drmanac et al, Science, 327: 78-81 (2010)), arrays of wells, which may include bead- or particle-bound templates (such as with 454, e.g. Margulies et al, Nature, 437: 376-380 (2005) or Ion Torrent sequencing, U.S. patent publication 2010/0137143 or 2010/0304982), micromachined membranes (such as with SMRT sequencing, e.g. Eid et al, Science, 323: 133-138 (2009)), or bead arrays (as with SOLiD sequencing or polony sequencing, e.g. Kim et al, Science, 316: 1481-1414 (2007)).

In another embodiment, such methods comprise amplifying the isolated molecules either before or after they are spatially isolated on a solid surface. Prior amplification may comprise emulsion-based amplification, such as emulsion PCR, or rolling circle amplification. These methods can also include Solexa-based sequencing where individual template molecules are spatially isolated on a solid surface, after which they are amplified in parallel by bridge PCR to form separate clonal populations, or clusters, and then sequenced, as described in Bentley et al (cited above) and in manufacturer's instructions (e.g. TruSeq™ Sample Preparation Kit and Data Sheet, Illumina, Inc., San Diego, Calif., 2010); and further in the following references: U.S. Pat. Nos. 6,090,592; 6,300,070; 7,115,400; and EP0972081B1; which are incorporated by reference.

In an embodiment, individual molecules can be disposed and amplified on a solid surface form clusters in a density of at least 10⁵ clusters per cm²; or in a density of at least 5×10⁵ per cm²; or in a density of at least 10⁶ clusters per cm². In another embodiment, sequencing chemistries are employed having relatively high error rates. In such embodiments, the average quality scores, produced by such chemistries are monotonically declining functions of sequence read lengths. In some embodiments, the decline corresponds to 0.5 percent of sequence reads have at least one error in positions 1-75; 1 percent of sequence reads have at least one error in positions 76-100; and 2 percent of sequence reads have at least one error in positions 101-125. The overall error rates vary and can be as low as 0.1% at every base position to every other base

In one or more embodiments, the methods of the present invention are able to detect mixtures of human DNA down to a LD of 0.01% (1 in 10,000) using haplotype counting by NGS, 100-fold more sensitive than current STR based methods. False positives from NGS are avoided by using haplotypes because if they vary from one another by enough SNPs, they should demonstrate no crosstalk, even with a mutation frequency of 0.1% to 1% per base. Although HLA-A was used as an illustrative proof-of-concept in the present invention, other haplotype loci from the 1000 Genomes database were identified using the inventive methods that can also be used for this purpose.

In accordance with some embodiments, selection of suitable loci may be influenced by the patient's ethnic background, number of discriminating SNPs and ease of primer placement. The set of haplotypes used for this purpose ideally would be suitable for transplant analysis of all patient ethnicities. Patient DNA is consistently detected bone marrow samples that test all donor by the conventional STR assay.

Detection of minimal residual disease (MRD) in cases such as acute promyelocytic leukemia (APL) and subsequent early intervention has been associated with significantly higher survival.

In accordance with an embodiment, the haplotype counting based assay of the present invention can be valuable to detect relapse in HSCT patients earlier than the existing microsatellite based assays. One of the other non-chromosome 6 (containing human HLA) loci (Table 1) could be better for this purpose, as one mechanism that leukemic cells can escape donor anti-leukemic T-cells is through the loss of the mismatched HLA allele, estimated to occur in 29.4 to 66.7% of such patients. Another limitation of the HLA-A haplotype approach is that some transplant donors are HLA-identical and loci other than HLA would be required to monitor such patients.

TABLE 1
Alternative loci for use with haplotype identification

| Chr* | Gene† | Start‡ | Stop | bp | #SNPs | #Haps | CEU | JPT | YRI | Mean |
|---|---|---|---|---|---|---|---|---|---|---|
| 1 | — | 238855189 | 238855428 | 240 | 9 | 7 | 0.63 | 0.46 | 0.63 | 0.57 |
| 1 | — | 238416454 | 238416673 | 220 | 9 | 11 | 0.39 | 0.46 | 0.43 | 0.43 |
| 2 | — | 106142506 | 106142791 | 286 | 10 | 8 | 0.63 | 0.61 | 0.66 | 0.63 |
| 2 | VIT | 36927840 | 36928071 | 232 | 9 | 6 | 0.58 | 0.59 | 0.61 | 0.59 |
| 3 | ST6GAL1 | 186654745 | 186655022 | 278 | 9 | 12 | 0.72 | 0.67 | 0.73 | 0.70 |
| 3 | — | 97907092 | 97907374 | 283 | 10 | 13 | 0.58 | 0.42 | 0.64 | 0.55 |
| 4 | SORCS2 | 7447136 | 7447434 | 299 | 9 | 16 | 0.60 | 0.46 | 0.65 | 0.57 |
| 4 | — | 66995978 | 66996254 | 277 | 9 | 8 | 0.63 | 0.45 | 0.60 | 0.56 |
| 5 | — | 125498526 | 125498753 | 228 | 10 | 19 | 0.67 | 0.58 | 0.67 | 0.64 |
| 5 | — | 178259629 | 178259892 | 264 | 13 | 23 | 0.69 | 0.56 | 0.64 | 0.63 |
| 6 | HLA-DRB1 | 32551493 | 32551765 | 273 | 9 | 306 | 0.98 | 0.95 | 0.96 | 0.96 |
| 6 | HLA-B | 31319390 | 31319668 | 279 | 10 | 27 | 0.93 | 0.91 | 0.88 | 0.91 |
| 7 | — | 64895079 | 64895355 | 277 | 10 | 12 | 0.59 | 0.61 | 0.66 | 0.62 |
| 7 | — | 9111002 | 9111297 | 296 | 9 | 11 | 0.60 | 0.64 | 0.59 | 0.61 |
| 8 | — | 6160141 | 6160442 | 302 | 10 | 13 | 0.56 | 0.67 | 0.63 | 0.62 |
| 8 | CSMD1 | 3478267 | 3478568 | 302 | 10 | 13 | 0.63 | 0.51 | 0.66 | 0.60 |
| 9 | — | 132197723 | 132197947 | 225 | 11 | 11 | 0.66 | 0.45 | 0.53 | 0.55 |
| 9 | — | 95691312 | 95691534 | 223 | 10 | 14 | 0.39 | 0.35 | 0.46 | 0.40 |
| 10 | — | 133376146 | 133376435 | 290 | 9 | 14 | 0.74 | 0.74 | 0.78 | 0.75 |
| 10 | — | 123095093 | 123095377 | 285 | 10 | 18 | 0.74 | 0.78 | 0.69 | 0.74 |
| 11 | CNTN5 | 99491238 | 99491538 | 301 | 9 | 7 | 0.62 | 0.50 | 0.60 | 0.57 |
| 11 | — | 5078880 | 5079122 | 243 | 10 | 15 | 0.65 | 0.40 | 0.61 | 0.55 |
| 12 | — | 82460264 | 82460531 | 268 | 9 | 7 | 0.65 | 0.66 | 0.66 | 0.66 |
| 12 | — | 17884472 | 17884719 | 248 | 9 | 10 | 0.67 | 0.61 | 0.66 | 0.65 |
| 13 | FARP1 | 99084002 | 99084259 | 258 | 9 | 17 | 0.63 | 0.70 | 0.71 | 0.68 |
| 13 | — | 33553507 | 33553784 | 278 | 9 | 29 | 0.68 | 0.45 | 0.65 | 0.59 |
| 14 | — | 107094021 | 107094317 | 297 | 10 | 25 | 0.53 | 0.51 | 0.66 | 0.57 |
| 14 | TRA | 22736235 | 22736521 | 287 | 9 | 11 | 0.51 | 0.57 | 0.61 | 0.56 |
| 15 | — | 34750054 | 34750330 | 277 | 9 | 8 | 0.51 | 0.57 | 0.61 | 0.56 |
| 15 | — | 25047399 | 25047681 | 289 | 10 | 25 | 0.51 | 0.44 | 0.55 | 0.50 |
| 16 | — | 56576389 | 56576687 | 299 | 11 | 46 | 0.76 | 0.82 | 0.80 | 0.79 |
| 16 | — | 84540653 | 84540932 | 280 | 9 | 16 | 0.69 | 0.66 | 0.74 | 0.70 |
| 17 | TBCD | 80804085 | 80804372 | 288 | 9 | 33 | 0.70 | 0.70 | 0.74 | 0.71 |
| 17 | RBFOX3 | 77148074 | 77148367 | 294 | 10 | 15 | 0.38 | 0.60 | 0.52 | 0.50 |
| 18 | — | 76597079 | 76597332 | 254 | 9 | 14 | 0.40 | 0.40 | 0.40 | 0.40 |
| 18 | CLUL1 | 631210 | 631400 | 191 | 9 | 12 | 0.35 | 0.27 | 0.38 | 0.33 |
| 19 | CCDC61 | 46500080 | 46500360 | 281 | 9 | 14 | 0.41 | 0.38 | 0.43 | 0.41 |
| 19 | — | 57525179 | 57525424 | 246 | 13 | 10 | 0.37 | 0.39 | 0.41 | 0.39 |
| 20 | PROKR2 | 5289890 | 5290161 | 272 | 9 | 7 | 0.64 | 0.51 | 0.59 | 0.58 |
| 20 | SIRPA | 1895467 | 1895674 | 208 | 11 | 24 | 0.49 | 0.58 | 0.56 | 0.54 |
| 21 | — | 20548907 | 20549196 | 290 | 9 | 6 | 0.44 | 0.35 | 0.42 | 0.40 |
| 21 | UMODL1 | 43528819 | 43529079 | 261 | 9 | 10 | 0.33 | 0.38 | 0.39 | 0.36 |

*Chromosome 22 is absent due to the lack of good polymorphic loci. X and Y excluded.
†Gene, if present.
‡Hg19 coordinates.

HSCT has traditionally been used to treat malignant and non-malignant hematologic disorders. In addition, with the transplantation of solid organs, some amount of lymphoid tissue may be transferred by cell migration from the donor organs, thereby creating chimerism in the patient. In contrast, intentional induction of microchimerism, by injecting donor BM, is a strategy used to create donor-specific tolerance in extremity transplantation. The development and persistence of donor-recipient microchimerism may be associated with the acceptance of transplanted organs.

As such, in accordance with another embodiment, the inventive methods with NGS to detect low levels of donor cells, provides an opportunity to document such microchimerism (or lack thereof) and to manage immunosuppressive regimens to optimize engraftment.

NGS of highly polymorphic regions using the inventive methods has applications outside of transplantation medicine. Microchimerism resulting from bi-directional exchange of cells between mother and fetus has been detected in women long after pregnancy using Y chromosome FISH and PCR for the SRY gene.

In accordance with some embodiments, the present invention can be applied for such studies with the additional benefit of also being able to detect exchanged cells between a mother and a daughter. NGS haplotyping using the inventive methods could be used to aid in the detection of rare tissue regenerative cells in the heart transplant setting. In sex-mismatched heart transplants and using Y chromosome FISH, studies have shown cardiac chimerism caused by migration of recipient cells to the grafted heart.

The field of forensics has relied upon STR loci for identification of suspects and human remains.

In accordance with another embodiment, testing haplotypes using the inventive methods can allow one to identify the presence of suspect in a large mixture of DNAs such as in “poly-suspect” cases. In addition to forensic applications, tools for quality control and sample tracking in large inventory of human DNA samples would be very valuable. Such tools have previously been reported using panels of SNPs, and the NGS-haplotyping assay of the present invention can be applied in similar situations. Similar markers could be developed to distinguish among species.

To ensure patient identity and exclude the possibility of a mislabeled specimen, in clinical laboratory settings, the present invention can be used to uniquely define the patient using one or more haplotypes, and then could easily be included in any NGS based genetic test. They can also be used for any absolutely critical test, such as ABO typing of blood products, and matched to the intended patient's genotype encoded in an implanted microchip immediately prior to transfusion. Although rare, wrong-patient adverse events occur in hospital settings, especially when patients share the same name or patients with similar appearances. To circumvent these preventable errors, a biological identifier, such as the haplotypes described in the present invention, could be implemented to unequivocally distinguish patients.

A kit is also provided comprising an array of oligonucleotides as described herein, or portions or fragments thereof, as well as a biochip as described herein, along with any or all of the following: assay reagents, buffers, probes and/or primers, and sterile saline or another pharmaceutically acceptable emulsion and suspension base. In addition, the kits may include instructional materials containing directions (e.g., protocols) for the practice of the methods described herein.

### EXAMPLES

Sample collection and preparation. Cell lines HCT116 (A*01:01:01 and A*02:01:01) and DLD-1 (A*02:01:01 and A*24:02:01:01) were chosen due to their available HLA-A haplotypes, and they were confirmed in the Immunogenetics Laboratory of the Johns Hopkins University, as well as their DNA fingerprint profile using the AmpFlSTRO Profiler Plus® PCR Amplification Kit (Life Technologies, Carlsbad, Calif.). A large number of cells were expanded to permit making cell dilutions, since we have found that cell dilutions are generally more accurate then DNA dilutions (data not shown). Cell to cell dilutions were made, where appropriate number of HCT116 cells was added to 10 million DLD-1 cells for each dilution. DNA was extracted from each cell pellet using DNeasy Blood and Tissue kit (Qiagen, Valencia, Calif.). Extracted DNA 1 was quantified by Quantifiler (Applied Biosystems, Carlsbad, Calif.) and stored at −20° C.

BME Samples. Samples from eighteen patients who underwent allogeneic HSCT were obtained from Molecular Diagnostic Laboratory at the Johns Hopkins Hospital, on an Institutional Review Board approved protocol. Samples were selected based on the disease type, the ability to distinguish patient from donor alleles, that they were all donor by STR analysis and that at least 600 ng (100,000 genomes) of DNA was available to test (Table 2). Each BM sample was prepared for sequencing as described below and sequenced on the Ion Torrent PGM to insure approximately 100,000 reads were obtained.

TABLE 2
BME patient characteristics and percent patient DNA detected.

| Pat # | Dis* | Patient HLA-A | Donor HAL-A | Diff ^{†} | % Pat |
|---|---|---|---|---|---|
| BME14 | AML | A*02:05:01:/A*03:01:01:01 | A*02:05:01:/A*01:01:01:01 | 11.6 | 0.000% |
| BME20 | MDA | A*29:02:01:01/A*26:01:01 | A*01:01:01:01/A*26:01:01 | 12.6 | 0.001% |
| BME18 | AML | A*03:01:01:01/A*30:01:01 | A*11:01:01/A*30:01:01 | 7.5 | 0.019% |
| BME10 | AML | A*32:01:01/A*34:02:01 | A*01:01:01:01/A*34:02:01 | 12.3 | 0.0.30% |
| BME19 | AML | A*24:02:01:01/A*68:01:01:01 | A*32:01:01/A*68:01:01:01 | 6.7 | 0.052% |
| BME25 | ALL | A*01:01:01:01/A*03:01:01:01 | A*33:01:01/A*03:01:01:01 | 12.11 | 0.060% |
| BME30 | AML | A*03:01:01:01/A*24:02:01:01 | A*03:01:01:01/A*01:01:01:01 | 7.6 | 0.136% |
| BME27 | AML | A*02:01:01:01/A*24:02:01:01 | A*02:01:01:01/A*02:01:01:01 | 7 | 0.140% |
| BME16 | AML | A*02:XX/A*26:XX | A*02:XX/A*03:XX | 6.9 | 0.189% |
| BME11 | AML | A*30:02:01/A*74:01 | A*68:02:01:01/A*74:01 | 8.2 | 0.190% |
| BME4 | AML-M6 | A*02:01:01:01/A*29:02:01:01 | A*02:01:01:01/A*68:01:01:01 | 6.5 | 0.312% |
| BME26 | ALL | A*11:01:01/A*30:01:01 | A*25:01:01/A*30:01:01 | 6.9 | 0.328% |
| BME1 | MM | A*02:01:01:01/A*23:01:01 | A*30xx/A*02:xx | 4.7 | 0.416% |
| BME22 | CLL | A*02:01:01:01/A*66:01 | A*01:01:01:01/A*66:01 | 11.9 | 0.640% |
| BME21 | MCL | A*68:02:01:01/A*29:02:01:01 | A*30:01:01/A*29:02:01:01 | 8.6 | 0.750% |
| BME29 | AML | A*11:01:01/A*26:01:01 | A*11:01:01/A*11:01:01 | 6 | 0.863% |
| BME23 | DLBCL | A*02:01:01:01/A*26:01:01 | A*02:01:01:01/A*02:01:01:01 | 9 | 1.110% |
| BME24 | HD | A*01:01:01:01/A*29:02:01:01 | A*30:02:01:/A*29:02:01:01 | 13.12 | 1.470% |

*Disease; DLBCL, diffuse large B-cell lymphoma; HD. Hodgkin's disease; MM, multiple myeloma.
^{†}Number of SNP differences between unique alleles (bold) and between the unique patient and shared alleles.

HLA-A PCR amplification. Forward and reverse primers were ordered with Ion Torrent-specific adaptors (A16 and P1-adaptors) added at their 5′ ends. Briefly, the first round PCR reaction included 600 ng (100,000 genomes) of DNA, 200 nM forward and reverse primers, in Platinum® PCR SuperMix High Fidelity (Invitrogen, Carlsbad, Calif.) in a total of 100 μl reaction volume. The forward primer was (5′-CCATCTCATCCCTGCGTGTCTCCGAC-tcag-AGGACCTGCGCTCTTGGAC-3′) (SEQ ID NO: 1), where A-adaptor (underlined), library key (lower case), and HLA-A primer (italics), and the reverse primer was (5′-CCTCTCTATGGGCAGTCGGTGAT-CGTTCTCCAGGTATCTGCGGA-3′) (SEQ ID NO: 2), including the P1-adaptor (underlined) and HLA-A reverse (italics). The expected size of the full length PCR product is 298 base pairs, including adapters. Following amplification, adaptor containing PCR product was visualized by gel electrophoresis (Novex® TBE Gels, Life Technologies, Carlsbad, Calif.) and quantified using High Sensitivity dsDNA reagents and Qubit 2.0 fluorometer (Invitrogen). With the desire to “paint” a single molecule on each bead for emulsion PCR, samples were diluted to a 0.03 nM working concentration. During emulsion PCR each single molecule produces a “clone” of progeny molecules (estimated to be ˜500,000) for sequencing.

Ion Torrent PGM library preparation and sequencing. Emulsion PCR using 20-25 μl working stock, next generation sequencing, and mapping to hg19 were all done per manufacturer's protocol (Life Technologies). Assessment of the percent amplicon containing beads was performed per manufacturer's protocol (Life Technologies) and measured with Qubit 2.0 fluorometer (Invitrogen, Carlsbad, Calif.). Amplicon coated beads were analyzed on 314 and 316 chips using the Ion PGM Sequencing 200 kit on the Life Technologies' Ion Torrent Personal Genome Machine™ (PGM), semiconductor sequencer, which detects dNTP incorporation using the hydrogen ion that is released (along with pyrophosphate) when a dNTP is incorporated into an elongating DNA strand (Nature 2011, 475:348-352 16; Electrophoresis 2012, 33:3397-3417).

Microsatellite analysis. PCR amplification of 9 microsatellites (AmpF1STRO Profiler® kit; Applied Biosystems) or 15 microsatellites (Identifiler®, Applied Biosystems) was performed according to the manufacturer's instructions. Amplicons were resolved on a capillary electrophoresis ABI3130x1 Genetic Analyzer (Applied Biosystems).

Bioinformatics/analysis. Initial processing of the data was done using the Ion Torrent platform-specific pipeline software Torrent Suite v3.2.1 to generate sequence reads, trim adapter sequences, and remove poor quality reads. Resulting sequence files were aligned to hg19 reference sequence. Further analysis of Fastq files was done using Geneious Pro 5.5.7 where perfectly matched reads were counted. Some samples were log10 transformed and graphed (GraphPad Prism v5.04).

Analysis of molecular specificity was done with homozygous HLA-A*01:01:01:01 and HLA-A*02:01:01:01 samples (hereafter simply A*01 and A*02, respectively). Each sample was analyzed for the other allele, followed by the introduction of the other allele's base at 11 SNP positions. For example, homozygous A*01 was analyzed for perfectly matched A*01 reads followed by perfect A*02 reads. Upon this, one A*02-specific base was introduced into A*01 sequence at each of the 11 SNP positions and analyzed. After introducing one A*02-specific base at all of the possible positions, 2 consecutive A*02-specific bases were introduced, repeating the process for 11 possible combinations. The process was continued all the way to substituting all 11 consecutive A*02-specific bases. The number of reads found, for each combination, was averaged and percent of reads found was graphed.

Bone marrow transplant samples were analyzed for the amount of patient (patient unique allele) in the sample. After eliminating reads representing a haplotype shared by both individuals, we calculated a percent patient DNA (patient/patient+donor). Cases where three alleles were shared, the equation [(2×unique patient)/(unique patient+shared patient-donor)] was used.

Bioinformatics analysis of 1000 Genomes database. Using 1000 Genomes release v3_20110521 from build hg18, we identified regions that were polymorphic and flanked by constant regions. We required a minimum of 9 variants with a MAF of ≧9% in three of the major populations: CEU, (CEPH, Utah Residents with Northern and Western European Ancestry); JPTCHB (a combined Asian population including JPT, Japanese in Tokyo, Japan, and CHB, Han Chinese in Beijing, Ching); and YRI (Yoruba in Ibadan, Nigeria) within 300 bp. A 300 bp size was chosen so that it could be amplified and sequenced in a single read, well within the limits of current NGS technology. We required potential regions to be flanked by constant regions of at least 20 bp for primer placement.

Polymorphic regions were converted from hg18 to hg19 using UCSC's LiftOver tool (genome.ucsc.edu/cgi-bin/hgLiftOver, last accessed Mar. 28, 2014). A custom script was written to convert phased variants from 1000 Genomes VCF files (both IMPUTE217 and SHAPEIT218 versions) into IMPUTE reference panel format haplotypes at every region. A custom program was then used to assess all possible haplotype combinations between two theoretical individuals for informativity and probability of occurrence. Particular haplotype combinations were defined as informative if at least one haplotype of individual B (patient) had at least 2 SNP differences from both haplotypes of individual A (donor). Once informative haplotype combinations were determined, the probabilities of these combinations occurring in two unrelated individuals was calculated (based on the 1000 Genomes frequencies) and summed Results from the two methods of phasing the chromosomes were highly correlated (data not shown), so only the data from SHAPEIT2 phased haplotypes are presented. Thus, the calculated probability score reflects the informativity of the region for 2 unrelated individuals.

### Example 1

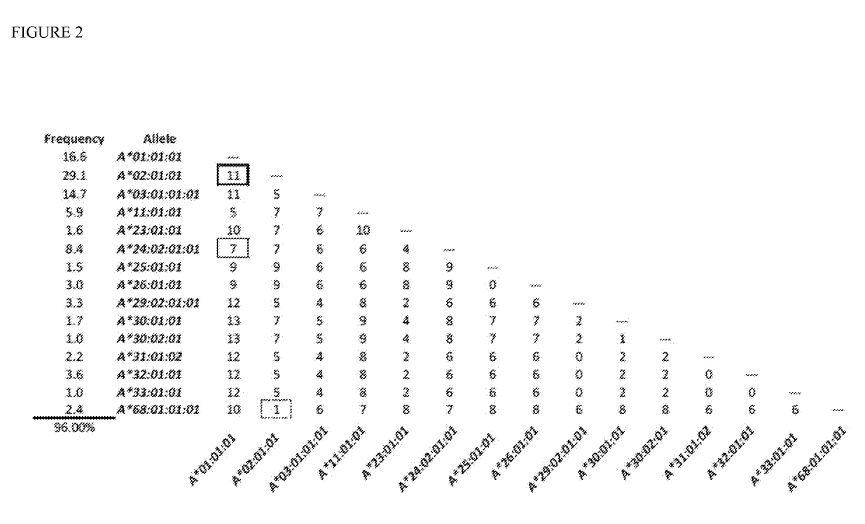

Analysis of HLA-A alleles. We performed alignments of common HLA-A alleles in the European-origin population using dbMHC database, publically accessible platform for DNA and clinical data related to the human Major Histocompatibility Complex (ncbi.nlm.nih.gov/gv/mhc/main.cgi?cmd=init last accessed Mar. 28, 2014). Regions with a high density of SNPs and flanked by non-polymorphic DNA were identified. One region, HLA-A exon 3 contained 18 possible SNPs and at least 15 major alleles in the European-origin population. We tested a series of primers surrounding this region and selected the best pair based on amplification efficiency and specificity (FIG. 1D, see methods). The number of SNP differences in this region between the most common alleles of the European-origin population was tabulated (FIG. 2). Some combinations of HLA-A alleles are easily differentiated whereas others are more difficult. For example, 11 SNPs differentiate allele A*01 from A*02 (double lined box), so that even with a relatively high base substitution error rate (e.g. 1% per base), a sample homozygous for A*01 should not contain any false positive A*02 allele reads. In contrast, A*02 and HLA-A*68:01:01:01 (hereafter A*68) have only 1 SNP difference (dashed lined box), so a pure sample homozygous for A*02 will likely contain reads matching A*68 due to the relative high error rate intrinsic to current NGS technologies.

### Example 2

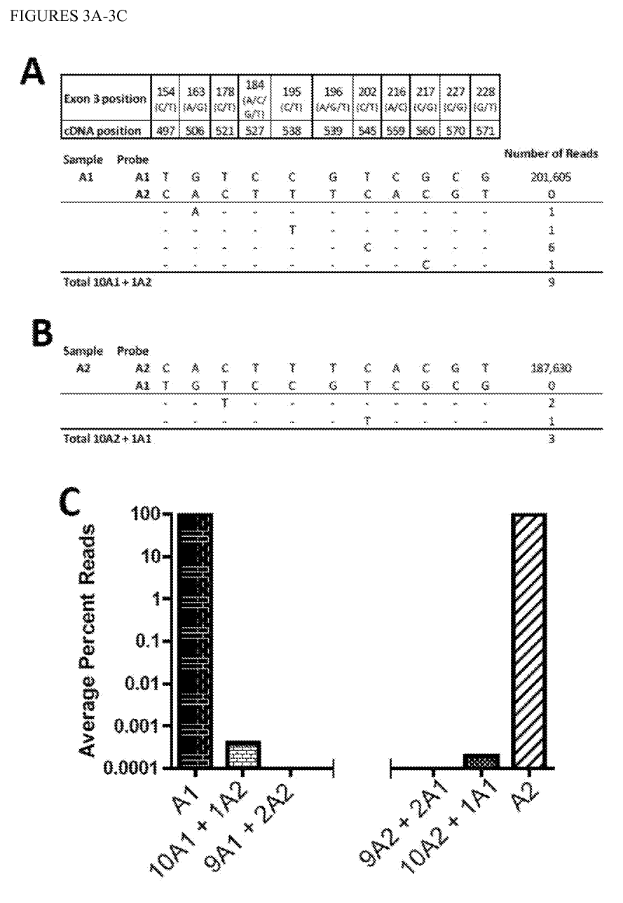

Determination of “crosstalk” between molecules which vary by 11 SNPs. To test this experimentally, we sequenced two samples, one homozygous for A*01 and another homozygous for A*02, and analyzed each for the other allele (FIG. 3). The A*01 and A*02 samples contained approximately 200,000, perfect matching reads. Neither pure sample contained any perfect reads of the other haplotype. We then examined the A*01 sample files for reads containing a single A*02 SNP at each of the 11 positions and found an average of 0.3-0.8% reads that contained a single error from the perfect haplotype (FIG. 3A). When two SNPs of the opposite haplotype were searched for, no reads were obtained. Similar results were obtained with the pure A*02 sample (FIG. 3B). A double waterfall plot shows that when enough discriminating SNPs between two individuals' alleles exist, the assay is highly specific. (FIG. 3C).

### Example 3

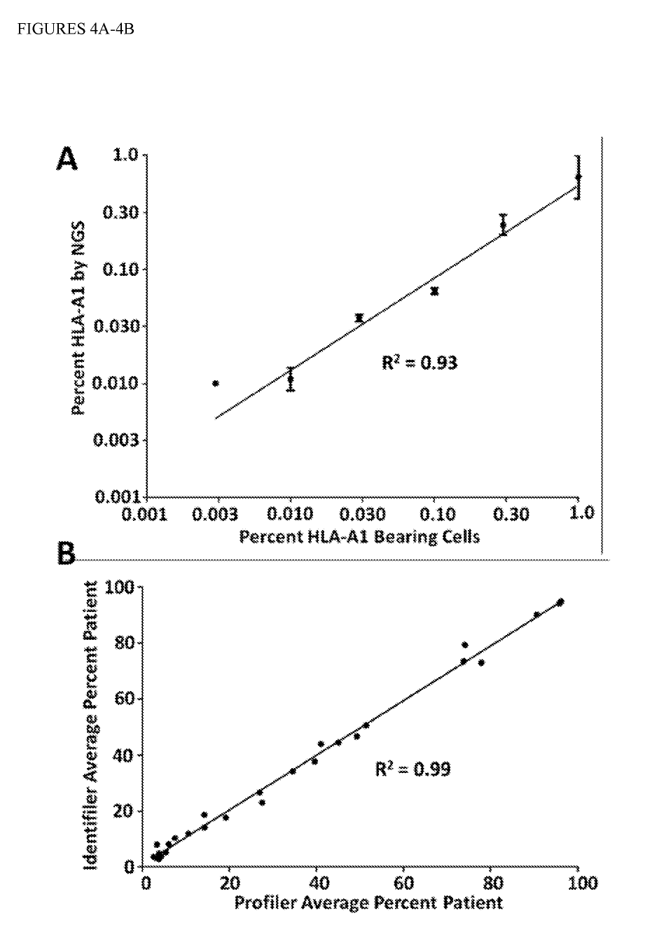

HLA-A dose-response curve, accuracy, precision, and limit of detection (LD). To assess the accuracy and LD, we generated a dilution series from two cell lines with known HLA-A genotypes. These samples were chosen because the two alleles of interest (A*01 and A*02) vary from one another by 11 SNPs and both vary from the commonly shared allele (A*24) by 7 SNPs (FIG. 2). Dilutions were made with cell mixes varying from 1 in 1 million (0.0001%) to 1 in 100 (1%) using a total of 10 million cells for each dilution. DNA was isolated and PCR performed using 600 ng of DNA. We chose this relatively large amount of DNA based on the desire to achieve a LD of at least 1:10,000 (0.01%) and to exceed that target LD by using 10x excess DNA. This relatively high DNA input reflects approximately 100,000 genomes (based on approximately 6 picograms/haploid genome) and was chosen to prevent “bottlenecking” and resultant allele dropout. For example, if DNA representing only 100 genomes were analyzed to 100,000× depth of coverage, the LD is input DNA limited, not depth of coverage limited, and is at best 1% (not accounting for Poisson sampling). A sample with a minor allele frequency of 0.1% would likely not be detected. Each sample was sequenced at least twice, and results graphed as the percent HLA-A*01 as a function of the percent HLA-A*01 bearing cells (FIG. 4A). Least-squares analysis generated a straight line demonstrating excellent accuracy (R2=0.93). To determine precision we performed additional replicates at 0.1% and 0.01% dilutions (4 total). The assay was highly precise at 0.1% cell mix (mean=0.065%, coefficient of variation =0.12), but less so at the 0.01% cell mix as expected (mean=0.012%, coefficient of variation=0.40). Because we detected the minor HLA-A allele in 4 out of 4 replicates at 0.01%, but only 1 out of 2 replicates at 0.003%, we concluded that 0.01% is the lower LD for this assay (data not shown). These results compare favorably with that of microsatellites, which are highly linear and generally accurate, but with a much poorer LD of only about 3% (FIG. 4B).

### Example 4

SNP haplotype assay detects patient DNA in bone marrow samples that tested all donor by STR analysis.

We selected 18 patients whose donor-patient HLA genotypes varied by at least 4 SNPs (Table 2). The allele unique to donor varied from that unique to patient by 4-13 SNPs.

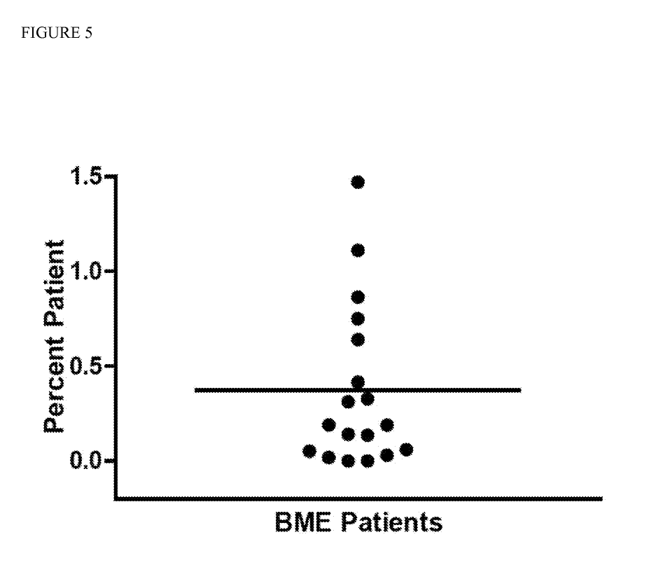

To further prevent false positive results, the unique patient allele varied from the shared allele by 2-12 SNPs. We also required that 600 ng be available for testing, so that the number of genomes (˜100,000) exceeded by an order of magnitude the desired LD. Several samples were excluded that could not meet these criteria. All samples tested positive for some level of patient DNA, except one. The positives ranged from 0.001% to 1.47% patient DNA (FIG. 5, mean 0.373), confirming that the haplotype based assay was more sensitive. We also noted that of the 4 samples with the highest levels of patient DNA, 3 of them were from patients with lymphoma.

### Example 5

The human genome contains clusters of SNPs that can be amplified to give information on haplotypes.

We used variant calls from the 1000 Genomes to identify regions containing at least 9 SNPs in all three populations (Europeans, Asians, and Africans, see Methods) within 300 bp. We further required these regions to be flanked by at least 20 bp of non-polymorphic DNA. The non-polymorphic regions provide potential primer sites, while the variable regions are suitable for haplotype counting. We identified 4,349 such genomic loci across the genome, requiring that the 9 or more variants have minor allele frequencies greater than 9% in all three populations (data not shown).

### Example 6

One concern is that a cluster of SNPs may not be that informative because it simply represents a high-frequency haplotype. For example, a haplotype that exists within a population at a 2% level and containing 20 SNPs, in conjunction with a second haplotype with the other base at each of the 20 positions present in 98% of the population, is a relatively uninformative marker. In contrast, a locus with 20 SNPs with 10 different haplotypes each of which is present at 10% in the population is highly informative.

Using the 4,349 regions identified as SNP dense, we analyzed the 1000 (1092) Genomes phased variant call files to calculate overall probability that two unrelated individuals would be different at each locus. Briefly, at each locus, a file was generated that contained all of the SNPs for each of the 2,184 (2×1092) alleles. We then determined the number of distinct alleles and the number of times each of them was represented. We then calculated all possible combinations of alleles within donors and recipients, and finally determined the combined probability that two unrelated individuals would be informative. The most informative loci from each chromosome are shown in Table 1. These putative alternate loci will be experimentally validated in large cohorts of patients, and experiments are currently in progress to do this.

### Example 7

Use of haplotype counting to produce quality control DNA-mix standards to be used in evaluating ultrasensitive mutation detection methods.

Low-level point mutation detection is problematic using next generation sequencing (NGS) due to high error rates of the current platforms. Nevertheless, detecting point mutations with accuracy and high sensitivity is clinically important. Our initial work (Debeljak et al, JMD 2014, 16:495-503) showed that our haplotype counting (HLA-A) method with NGS was highly sensitive and accurate, thus providing a way to overcome intrinsically high error rates of NGS. Using this approach, we now demonstrate its utility to produce quality control samples that can be used to assess various ultrasensitive assays/technologies. To demonstrate this, we produced a dilution series, quality controlled it using the HLA-A haplotype counting method and then tested these controls using the AmpliSeq Cancer panel (FIG. 1A) and digital droplet PCR (ddPCR, FIG. 1B) technologies.

### Example 8

Ability to detect low levels of donor bone marrow in the setting of limb transplantation.

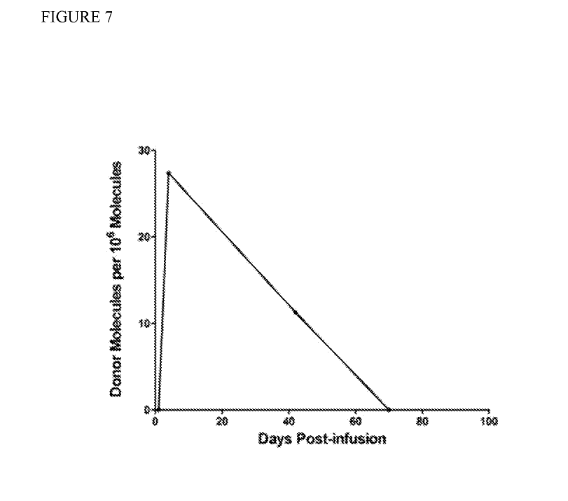

A patient underwent double arm transplantation, followed by infusion 2 weeks later of donor bone marrow (BM) cells. We evaluated various samples (PB, CD3, BM) at different time points post-infusion to determine whether donor-derived DNA can be detected. Donor-derived DNA was detected in PB samples at 0.003% and 0.001% in early days post-infusion (4 and 42 days, respectively, Table 3, FIG. 7) using the haplotype counting method. These samples all tested as donor-negative by the conventional STR assay.

TABLE 3
Samples tested and percent donor DNA detected.

| | | Days | | NGS Results | | | |
|---|---|---|---|---|---|---|---|
| | | Post | | Patient | Donor | Shared | |
| | | BM | STR | Unique | Unique | (A*02:05/ | % |
| Sample Alias | Type | Infusion | Results | (A*01) | (A*24) | A*2) | Donor |
| ArmTransplant Case 1-S1 | PB | 1 | 0% Donor | 5,352 | 0 | 2,117 | 0% |
| ArmTransplant Case 1-S2 | PB | 4 | 0% Donor | 804,061 | 22 | 328,699 | 0.003% |
| ArmTransplant Case 1-S7 | PB | 42 | 0% Donor | 1,331,462 | 15 | 1,218,256 | 0.001% |
| ArmTransplant Case 1-S9 | PB | 70 | 0% Donor | 18,311 | 0 | 48,929 | 0% |
| ArmTransplant Case 1-S10 | PB | 165 | 0% Donor | 13,900 | 0 | 37,420 | 0% |
| ArmTransplant Case 1-S11 | PB | 252 | 0% Donor | 1,523,636 | 0 | 1,519,307 | 0% |
| ArmTransplant Case 1-S16 | PB | 564 | 0% Donor | 1,544,569 | 0 | 1,216,153 | 0% |

### Example 9

Ability to predict early relapse of AML patients after bone marrow transplantation.

Currently, relapse of AML post BM transplantation is tested by STR analysis, but this technology is relatively insensitive (limit of detection ˜3%). We have applied our haplotype counting assay to determine if we can detect relapse earlier in patients who underwent haploidentical transplants. A total of 15 patient samples (7 control/non-relapse and 8 relapse) were evaluated and results compared for significance. Samples evaluated in control group were on average 291 days post transplantation. Relapse samples evaluated were taken 53-145 days prior to STR-relapsed sample (see Table 4 below).

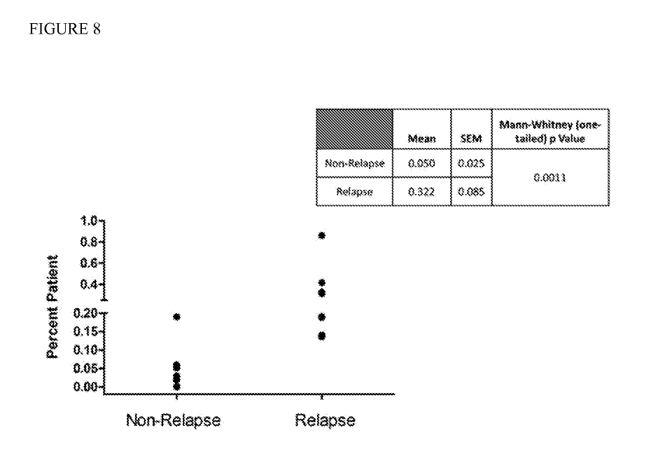

In controls (non-relapse), average patient-derived DNA was present at 0.05%. In contrast, average patient-derived DNA in relapse group was present at 0.32%. There is statistical significance between the two groups (FIG. 8, p=0.0011).

TABLE 4
Evaluation of AML Relapse post-BM transplantation

| | | | | | Days | | |
|---|---|---|---|---|---|---|---|
| | | | | Days | clean | | |
| | | | Days | pre- | after | Percent | |
| | | Number | post- | STR | our | (%) | |
| Patient ID | Disease | Differences^{a} | transplant | relapse | sample | Pre | |
| Non-Relapse Patients | | | | | | | |
| BME 10 | AML | 12.3 | 150 | — | 1354 | 0.03% | Average % Pre: 0.05% |
| BME 11 | AML | 8.2 | 180 | — | 354 | 0.19% | Avg Days Post Transplant: 291.1 |
| BME 14 | AML | 11.6 | 360 | — | 770 | 0.00% | |
| BME 18 | MDS/AML | 7.5 | 365 | — | —^{b} | 0.019% | |
| BME 19 | AML | 6.7 | 423 | — | —^{b} | 0.52% | |
| BME 20 | MGS | 12.6 | 378 | — | 721 | 0.001% | |
| BME 25 | ALL | 12.11 | 182 | — | 545 | 0.06% | |
| Relapse Patients | | | | | | | |
| BME 1 | IgG | 4.7 | 56 | 130 | — | 0.416% | Average % Pre: 0.322% |
| | multiple | | | | | | Avg Days Post Transplant: 38.5 |
| | myeloma | | | | | | |
| BME 4 | Acute | 6.5 | 60 | 50 | — | 0.312% | |
| | erythroid | | | | | | |
| | leukemia | | | | | | |
| BME 16 | AML | 6.9 | 60 | 111 | — | 0.389% | |
| BME 26 | Refractory | 6.9 | 179 | 145 | — | 0.328% | |
| | acute | | | | | | |
| | lymphobiatic | | | | | | |
| | leukemia | | | | | | |
| BME 27 | Secondary | 7 | 56 | 131 | — | 0.14% | |
| | AML | | | | | | |
| BME 29 | High risk | 6 | 94 | 130 | — | 0.853% | |
| | AML | | | | | | |
| BME 30 | AML- | 7.6 | 63 | 53 | — | 0.136% | |
| | Trisomy 8 | | | | | | |
| | with neg | | | | | | |
| | FLT-3 | | | | | | |
| BME 31 | Refractory | 2.5 | 141 | 129 | — | 0.191% | |
| | multiple | | | | | | |
| | myeloma | | | | | | |

^{a}Number of SNP differences between unique alleles
^{b}Two patients trasnferred and sample used in our assay was the last sample collected at our institution.

### Example 10

### Detecting Maternal Microchimerism in Fetal Tissues

We evaluated the presence of maternal microchimerism in various fetal tissues for 2 different cases. Each sample was also evaluated by STR (Identifiler) assay, which was unable to detect maternal DNA. Using our NGS-based haplotyping counting approach, we were able to detect maternal DNA in various fetal tissues (see Table 5). Quantity of the DNA used for testing varied from 140 ng (23,000 genomes) to 600 ng (100,000 genomes). Samples in each case were barcoded, pooled, enriched, and sequenced on a 316v2 chip using 400 bp chemistry kit.

TABLE 5
Detection of Maternal and Fetal DNA

| | Sample | STR | NGS |
|---|---|---|---|
| Case 1 | Case 1-Placenta | No maternal | 99.4% Fetus; |
| | | DNA detected | 0.60% Mother |
| | Case 1-Spleen | No maternal | 100% Fetus |
| | | DNA detected | |
| | Case 1-Lung | No maternal | 99.99% Fetus; |
| | | DNA detected | 0.01% |
| | | | Mother |
| | Case 1-Thymus | No maternal | 100% Fetus |
| | | DNA detected | |
| | Case 1-Liver | No maternal | 100% Fetus |
| | | DNA detected | |
| Case 2 | Case 2-Spleen | No maternal | 100% Fetus |
| | | DNA detected | |
| | Case 2-Lung | No maternal | 100% Fetus |
| | | DNA detected | |
| | Case 2-Thymus | No maternal | 99.99% Fetus; |
| | | DNA detected | 0.01% Mother |
| | Case 2-Liver | No maternal | 100% Fetus |
| | | DNA detected | |
| | Case 2-Lymph | No maternal | 100% Fetus |
| | Node | DNA detected | |

### Example 11

### Validation of Additional Loci

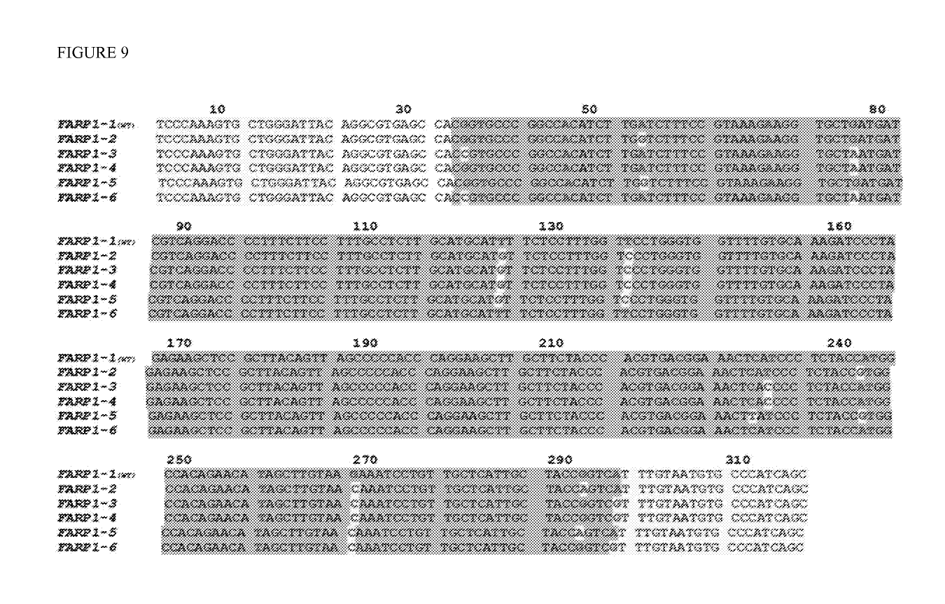

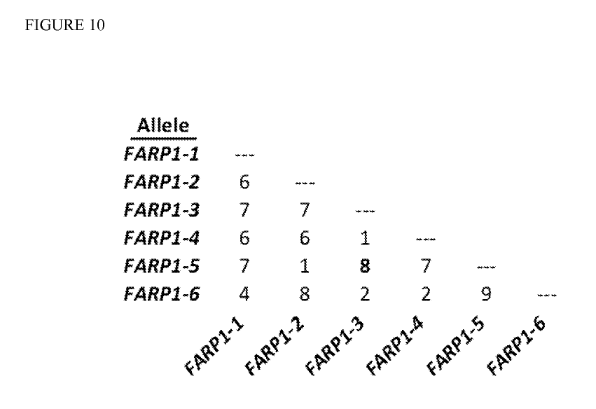

Previously (Debeljak et al, JMD 2014, 16:495-503), the present inventors bioinformatically discovered over 4,000 additional loci in the human genome that can be used for our haplotype counting assay. This is of value since a given transplanted patient may be identical to their donor at the HLA-A locus, or possess HLA-A alleles with too few SNPs that are different between the donor and recipient. The present inventors are working on experimentally validating some of the 4,000 additional loci. Table 6 shows some of the loci we are validating. Using an independent set of 8 normal samples, and using whole genome sequencing, we provide an alignment of FARP1 as an example (FIG. 9), and the haplotypes of FARP1 locus that were compared (FIG. 10).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for identifying informative haplotypes useful for identity testing comprising:
a) obtaining the DNA sequences of a plurality of individual genomes of a mammal;
b) identifying within the genomes of a) haplotypes comprising both allelic DNA sequences of about 100 to 400 or more base pairs in length, having at least one or more polymorphic regions which are flanked at both the 5′ and 3′ ends with constant regions of at least about 20 base pairs in length;
c) identifying within the haplotypes of b) those haplotypes which have at least about 2 or more single nucleotide polymorphism variants of the polymorphic regions;
d) identifying those haplotypes of c) as informative if at least 1 haplotype from a first individual genome has at least 2 or more single nucleotide polymorphism differences from both alleles of a second individual genome; and
e) preparing PCR primers using the constant regions identified in b).

2. The method of claim 1, wherein the DNA sequences of a plurality of individual genomes is from humans.

3. The method of claim 1, wherein the haplotypes of b) are 300 base pairs in length.

4. The method of claim 1, wherein in c), identifying within the haplotypes of b) those haplotypes which have at least about 2 or more single nucleotide polymorphism variants of the polymorphic regions

5. The method of claim 4, wherein the haplotypes of b) are located within a gene, or an intron region within a gene, or intragenic regions of the genomic DNA.

6. A method for determining the likelihood the presence of donor DNA sequence of one or more informative haplotypes in a DNA sample of a mammal which received donor cells comprising:
a) obtaining a sample containing a sufficient amount of DNA which comprises at least about 100,000 genomes of the mammal which received donor cells;
b) purifying the DNA from a);
c) amplifying the DNA from b) using PCR and primers and probes specific for one or more informative haplotypes;
d) analyzing the plurality of DNA sequences of the amplified informative haplotypes for single nucleotide polymorphisms in c);
e) comparing the DNA sequence single nucleotide polymorphisms found in d) to the DNA sequence single nucleotide polymorphisms for one or more reference informative haplotypes of the donor, wherein when a DNA sequence of the one or more informative haplotypes from d) does not contain all of the single nucleotide polymorphisms of the one or more reference informative haplotypes, the DNA sequence is discarded as erroneous;
g) establishing that when a DNA sequence of the one or more informative haplotypes from d) contains all of the single nucleotide polymorphisms of the one or more reference informative haplotypes of the donor, the DNA sequence is a match and the haplotype identity is confirmed; and
h) identifying that the donor cells engrafted in the mammal which received the donor.

7. The method of claim 6, wherein the donor cells are human stem cells.

8. The method of claim 6, wherein the donor cells are human bone marrow cells.

9. The method of claim 8, wherein the mammal receiving the donor cells is undergoing an organ transplant.

10. A method for identifying the presence of a suspect DNA sequence of one or more informative haplotypes in a sample comprising a mixture of DNA of a plurality of subjects comprising:
a) obtaining a sample containing a sufficient amount of DNA which comprises at least about 100,000 genomes of the DNA of a plurality of subjects;
b) purifying the DNA from a);
c) amplifying the DNA from b) using PCR and primers and probes specific for one or more informative haplotypes of the suspect DNA;
d) analyzing the plurality of DNA sequences of the amplified informative haplotypes for single nucleotide polymorphisms in c);
e) comparing the DNA sequence single nucleotide polymorphisms found in d) to the DNA sequence single nucleotide polymorphisms for one or more suspect informative haplotypes, wherein when a DNA sequence of the one or more suspect haplotypes from d) does not contain all of the single nucleotide polymorphisms of the one or more reference informative haplotypes, the DNA sequence is discarded as erroneous;
g) establishing that when a DNA sequence of the one or more informative haplotypes from d) contains all of the single nucleotide polymorphisms of the one or more suspect informative haplotypes, the DNA sequence is a match and the haplotype identity is confirmed; and
h) identifying that the suspect DNA is present in the sample.


SEQUENCE LISTING (2 sequences)

SEQ ID NO: 1 - DNA, 49 bp, Artificial Sequence
  synthetic sequence
ccatctcatccctgcgtgtctccgactcagaggacctgcgctcttggac

SEQ ID NO: 2 - DNA, 44 bp, Artificial Sequence
  synthetic sequence
cctctctatgggcagtcggtgatcgttctccaggtatctgcgga